(19) United States
(12) Patent Application Publication
(10) Pub. No.: US 2026/0321373 A1
(43) Pub. Date: Oct. 15, 2026

(54) Multi-echo fMRI reports

(71) Applicant: Ceretype Neuromedicine, Inc., Cambridge, MA (US)

(72) Inventors: Prantik Kundu, Flushing, NY (US); Roni Setton, Cambridge, MA (US); David Silbersweig, Chestnut Hill, MA (US); Emily Stern, Chestnut Hill, MA (US)

(21) Appl. No.: 19/484,864

(22) PCT Filed: May 17, 2024
(86) PCT No.: PCT/US2024/030084

§ 371 Date: Nov. 15, 2025

Related U.S. Application Data
(60) Provisional application No. 63/467,678, filed on May 19, 2023.
(60) Provisional application No. 63/599,510, filed on Nov. 15, 2023.
(60) Provisional application No. 63/554,964, filed on Feb. 17, 2024.

(51) Int. Cl. G16H 15/00; G16H 30/20; G16H 30/40; G16H 50/20

(52) U.S. Cl. CPC G16H 15/00; G16H 30/20; G16H 30/40; G16H 50/20

(57) ABSTRACT

Systems and methods for generating reports using multi-echo fMRI data. The method includes receiving multi-echo fMRI data; processing, using a processor, the multi-echo fMRI data to generate at least one feature, the at least one feature being a representation of the subject's functional network in R2*-based units, a functional network map, a bulk activation metric, a functional identity, a patient-specific functional architecture, a report identifier, or a quality control metric; generating, using the processor, a report using the at least one generated feature; and presenting, via a user interface, the generated report.

### CROSS REFERENCE TO RELATED APPLICATIONS

The present application claims the benefit of and priority to co-pending U.S. provisional application No. 63/467,678, filed on May 19, 2023, co-pending U.S. provisional application No. 63/599,510, filed on Nov. 15, 2023, and co-pending U.S. provisional Application No. 63/554,964, filed on Feb. 17, 2024, the entire content of each of which is hereby incorporated by reference as if set forth in its entirety herein.

### TECHNICAL FIELD

Embodiments described herein generally relate to systems and methods for generating reports and, more specifically but not exclusively, to systems and methods for generating reports for multi-echo fMRI data.

### BACKGROUND

Functional neuroimaging involves the measurement of brain activity. Functional neuroimaging is suited to numerous applications, including studying normal brain function, studying the action of a drug, differentiating a patient group from a control group to identify the pathophysiology of a disease, and mapping the metabolic state of brain regions generally with anatomical and temporal specificity.

Functional MRI (fMRI) scans the brain in slices, repeatedly, to reconstruct 3-dimensional volumes of the brain sequentially to track changes in brain activity over time. For each repetition, a radiofrequency pulse is applied to excite neural tissue, and images are acquired to capture signal across brain tissue as it decays.

fMRI differs from structural MRI (sMRI). fMRI yields time varying 3D data (i.e., very large data sets of 4-dimensional data) while sMRI yields 3D or 2D data.

Blood oxygenation level dependent (BOLD) fMRI captures changes in deoxygenated blood which directly reflect metabolic activity across the brain encoding numerous, ongoing neural processes. fMRI rapidly captures state and trait changes of the brain in response to external and internal stimuli, disease, and effects of drugs and treatment. In contrast, sMRI may not show any alterations, and if it does, this may require far longer exposure to a state to show any informative changes.

Significant work has also shown that in many cases sMRI lends little or no value to a study since the variables of interest, such as the volume of a region of interest, may be equivalent to structural variability across subjects. In contrast, fMRI may be analyzed using numerous methods for any temporal, spatial, or joint-spatiotemporal process, all of which may be elucidated by distinct or overlapping analysis techniques, including machine learning in supervised, semi-supervised, or potentially self-supervised methods.

Commensurate with the difference in the nature of data between sMRI and fMRI, the nature of reporting is distinct as well. sMRI reports may communicate brain regional or compartment values on a cross-sectional or longitudinal basis. In contrast, fMRI communicates numerous representations of functional state ranging from increased activity, which is a reflection of metabolic drive, to connectivity, which is a representation of the functional communication between regions throughout the brain, on either a point-to-point basis or a source-separated basis.

An important extension to standard fMRI methodology has been the development of multi-echo fMRI (ME fMRI). Standard single-echo fMRI acquires signals over space and time, which is sufficient if the temporal signature of an effect of interest is well defined and predictable. However, if no signature is known a priori, then heuristics are required to identify effects which are highly susceptible to errors of interpretation and analysis. This can be problematic as there are many sources of artifact affecting fMRI data overlapping substantially in spatial and temporal character (e.g., spectral envelope) with signals from neurally-related processes.

In ME-fMRI, three or more images are acquired at multiple echo times (TE) in the same time window to capture signal across the decay period. A distinguishing aspect of ME-fMRI is that the additional information can be used to systematically disentangle neuronal-related signals from non-neuronal artifactual signals based on the biophysical model of signal decay itself, and reporting processes and requirements should address these differences.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

According to one aspect, embodiments relate to a method for generating reports using multi-echo fMRI data. The method includes receiving multi-echo fMRI data; processing, using a processor, the multi-echo fMRI data to generate at least one feature, the at least one feature being a representation of the subject's functional network in R2*-based units, a functional network map, a functional identity, a patient-specific functional architecture, a report identifier, or a quality control metric; generating, using the processor, a report using the at least one generated feature; and presenting the generated report.

In some embodiments the multi-echo fMRI data is received from an imaging device.

In some embodiments the multi-echo fMRI data is retrieved from a storage device.

In some embodiments the method further includes preprocessing the multi-echo fMRI data to improve its usability.

In some embodiments the functional network map is color coded to reflect specific brain states.

In some embodiments the report identifier includes a bar code.

In some embodiments the quality control metrics include a temporal signal-to-noise or contrast-to-noise ratio.

In some embodiments, the generated report is presented via a static report or user interface.

According to another aspect, embodiments relate to a system for generating reports using multi-echo fMRI data. The system includes a communication interface for receiving multi-echo fMRI data; a user interface; and a processor configured to process the multi-echo fMRI data to generate at least one feature, the at least one feature being a representation of the subject's functional network in R2*-based units, a functional network map, a bulk activation metric, a functional identity, a patient-specific functional architecture, a report identifier, or a quality control metric; generate a report using the at least one generated feature; and present via the user interface the generated report.

In some embodiments the system further includes an imaging device.

In some embodiments the system further includes a storage device for storing multi-echo fMRI data.

In some embodiments the processor is further configured to preprocess the multi-echo fMRI data to improve its usability.

In some embodiments the generated functional network map is color coded to reflect specific brain states.

In some embodiments the generated report identifier includes a bar code.

In some embodiments the generated quality control metrics include a temporal signal-to-noise ratio.

According to still another aspect, embodiments relate to a non-transitory computer readable medium storing instructions that are executable by a processing device, and upon such execution cause the processing device to perform operations including receiving multi-echo fMRI data; processing, using a processor, the multi-echo fMRI data to generate at least one feature, the at least one feature being a representation of the subject's functional network in R2*-based units, a functional network map, a bulk activation metric, a functional identity, a patient-specific functional architecture, a report identifier, or a quality control metric; generating, using the processor, a report using the at least one generated feature; and presenting, via a user interface, the generated report.

In some embodiments the operations further comprise receiving multi-echo fMRI data from an imaging device.

In some embodiments the operations further comprise retrieving multi-echo fMRI data from a storage device.

In some embodiments the operations further comprise preprocessing the multi-echo fMRI data to improve its usability.

In some embodiments the functional network map is color coded to reflect specific brain states.

In some embodiments the report identifier includes a bar code.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 depicts an exemplary apparatus for generating multi-echo fMRI reports in accord with the present disclosure;

FIG. 2 presents an exemplary multi-echo fMRI report in accord with the present disclosure; and

FIG. 3 is a flowchart of a one embodiment of a method for generating multi-echo fMRI reports in accord with the present disclosure.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including standard hard drives, solid state storage, floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

Reference will now be made in detail to the exemplary embodiments, examples of which are illustrated in the accompanying drawings. Wherever possible, the same reference numbers will be used throughout the drawings to refer to the same or like parts.

Embodiments herein relate to the automated analysis and generation of reports from multi-echo fMRI data. Clinical reporting of task-based or resting state neural function imaged using multi-echo fMRI may confer significant value to the assessment of the functional state of a brain being imaged, which may be either human or non-human, and lead to significantly different reporting processes and requirements compared with standard fMRI, including differences in relevant metrics, units of expression, and quality.

Although embodiments disclosed herein discuss R2*-based units, which are time-varying fluctuations in radiological units of magnetic susceptibility, discussed in more detail in co-pending U.S. provisional application No. 63/599,510, and co-pending U.S. provisional application No. 63/554,964, embodiments may also utilize other metrics, including but not limited to T2* units and other similar units in existence now or developed in the future.

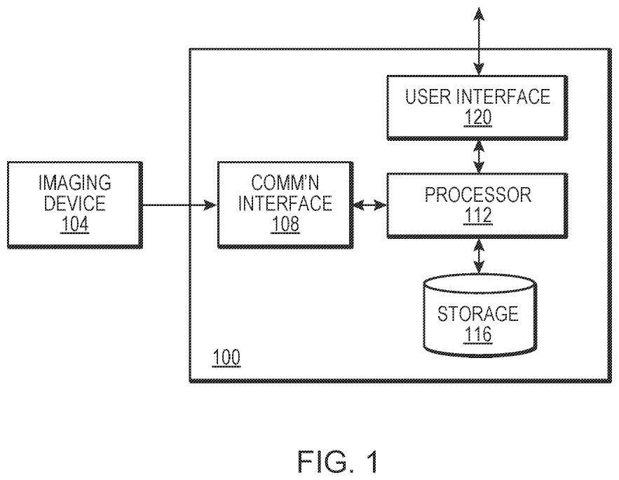

FIG. 1 is a block diagram of one embodiment of a report generating system 100. The system 100 includes an imaging device 104, a communication interface 108, a processor 112, a storage 116, and a user interface 120. In some embodiments, different components may be combined in a single device, such as a computer that includes a processor and storage, or they may each be their own separate device. In some embodiments one or more components may be implemented in a distributed system, with the imaging device located in, e.g., a hospital, and the processor and storage located in, e.g., a data center or cloud computing environment.

The imaging device 104 is typically an MRI scanner that uses one or more magnetic fields, magnetic field gradients, and radio waves to generate images of the organs in the body. In accord with the disclosure, the imaging device 104 is typically collecting images of a brain or brain region while a user performs one or more tasks of interest (i.e., fMRI). The activity changes the magnetism of the hemoglobin in the brain which can be viewed and analyzed in the collected imagery.

The communication interface 110 sends and receives data between the processor 112 and one or more external devices using a wired or wireless connection. As illustrated in FIG. 1, communication interface receives medical imaging data from the imaging device 104 and delivers it to the processor 112. The processor 112 may be a general-purpose microprocessor or a specialized device such as a digital signal processor or an application specific integrated circuit. A general-purpose microprocessor may implement the functionality discussed herein by way of executable programs stored on computer-readable media.

The processor 112 may process the data from the interface 110 to generate a diagnostic report based on the fMRI imaging collected by the imaging device 104 or it may deliver the fMRI data to storage 116 for later retrieval and analysis. The diagnostic report may be presented to an operator by way of the user interface 120, which may be a web browser, smartphone, or dedicated terminal.

The processor 112 may pre-process the imaging data by, e.g., filtering it to reduce noise and artifacts and performing other operations designed to improve the usability of the images, such as resizing and contrast adjustment, prior to presenting it via the user interface 120.

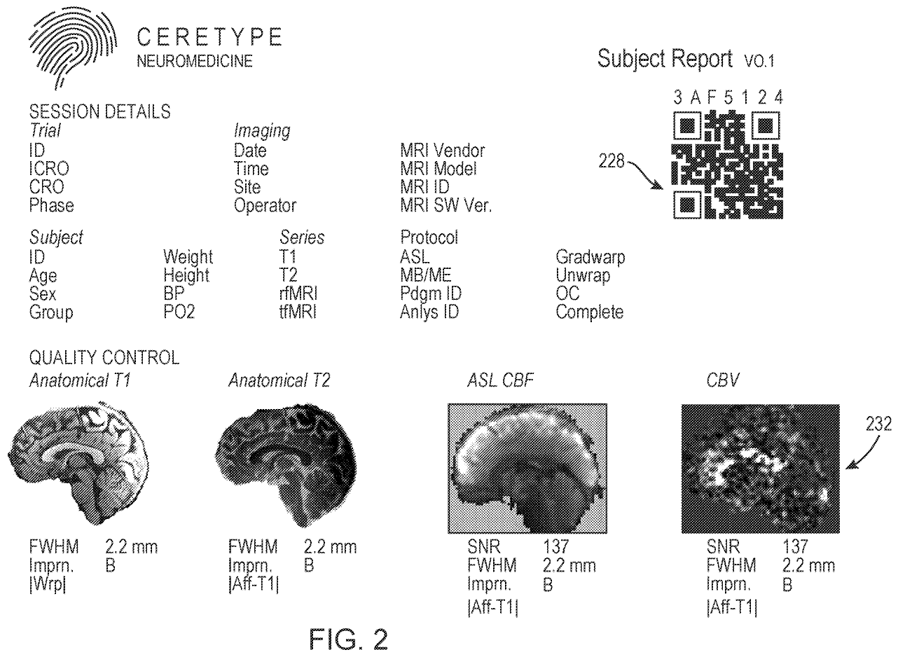

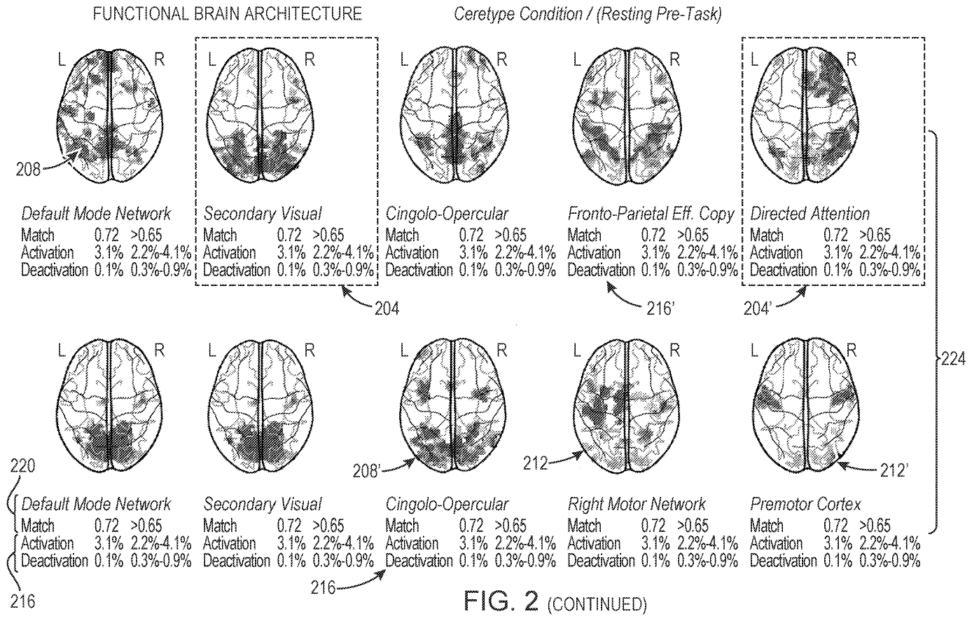

Reports generated from multi-echo fMRI in accord with this disclosure are uniquely reflective of behavioral, state, and trait aspects of the neurobiological systems being imaged in accord with this disclosure. These reports include one or more features, which may vary according to patient or operator choice. These features are explained below with respect to an exemplary report included as FIG. 2.

The first feature is that the subject's functional network is represented in R2*-based units by projection from a population-based network extracted from multi-echo fMRI data in a standard space (204^{N}). The report represents functional networks against various anatomical references, an individual's own unique anatomy, or a population average/representative brain image that is mapped to an atlas of brain regions. The capability to perform this registration in the clinical context where subject anatomy could vary significantly from the population norm is facilitated by the multi-echo fMRI measure of baseline R2*, which gives anatomical-functional co-registration guideposts for registration based directly on measures of functional relevance distribution from points throughout the brain.

The second feature is the presentation of a distinct functional network map corresponding to a distinct neurobiological process at the patient level (208^{N}). Color coding may be used to represent relaxometric parameter maps reflecting specific brain states, such as a resting state, a state of increased activity, or a state of decreased activity. In one embodiment, red areas represent increased activity or metabolic drive in response to a functional demand. In multi-echo data, this information uniquely communicates with metrics for reliability and is expressed in physical units (R2*-based units (ms)) that are internally consistent across neurobiological states such as drug or treatment/placebo, and/or instrumental conditions such as different MRI scanners.

The third feature is the juxtaposition of increased activity and decreased activity states (212^{N}). In one embodiment, areas in a first color represent increased activity and areas in a second color represent decreased activity or metabolic suppression to facilitate a functional response requiring mitigated function of the network. In multi-echo data, this information can uniquely have its amplitude of activation and suppression/deactivation be qualified in physical units (e.g. (+) or (−)ΔR2* units (ms)) that have features of consistency shared with the increased activity, and determine validity of effects that is not dependent on effect size, and is instead based on relaxometric markers that differentiate neural processes from nuisance effects. In multi-echo fMRI reports, this separation of effect amplitude and effect significance is facilitated by the information arising from independent aspects/dimensions of the underlying data, whereas in standard fMRI both are determined from the same data. This is important because deactivations conventionally arise as lower magnitude effects compared to activation effects, meaning without an independent thresholding mechanism, effects are biased toward increased activity. This is problematic from a clinical perspective as both increased activity and decreased activity are key aspects of brain function that may be markers of drug effects, disease, or behavioral conditions separately and even more so jointly.

The fourth feature is the display of bulk activation metrics (216^{N}). The comprehensive nature of the identified signal allows networks ascertained from multi-echo fMRI data to have their levels of increased activity and decreased activity represented by a summary statistic that reflects overall metabolic demand of a neural process, or measured separately for increased activity and decreased activity. Moreover, standard ranges are computed for these metrics which may be derived from a clinically normal range so that abnormal values may be gauged. This assessment may be conducted on the functional networks derived a priori from the data, representing an individual person uniquely, or as the projection of a population level network representation on to the data of an individual.

The fifth feature is the display of functional identity (220^{N}). The functional networks that arise from an individual in healthy and/or disease states have various degrees of repeatability and recognizability in reference to patterns derived from population level data. Such networks are associated with their functions by lookup in meta-analytic databases. Such look-ups, by virtue of their expression in standard units of activation (R2*-based units(ms)) or significance (F-statistics of multi-echo scaling) and presence at sample level leads to high reliability of match on the basis of data characteristics alone. Thus, the degree of match between a network arising from an individual and the population-level consensus network becomes informative as a factor of confluence or dissonance from a standard set.

The sixth feature is the display of patient-specific functional architecture (224). A representation of the set of neural processes spatially localized to their substrate tissues is estimated on the basis of an individual subject or patient a priori, or on the basis of spatial and/or temporal priors to focus network characterization onto brain processes of interest. This may represent but is not limited to a map of R2*-based magnitude measures in regions derived from population-based consensus networks, or a network map that is not limited to pre-defined regions and/or is derived from other analytic approaches to represent measures of significance. In multi-echo fMRI data, the count and type of functional networks can be reliably ascertained (along with artifacts) as a function of drug/state/trait factors, explaining the vast majority of data variance, meaning there is almost no functional information in the residual (which is mostly comprised of thermal noise).

The seventh feature is the report identifier (228). Multi-echo fMRI data offers a high degree of reproducibility due to its quantitative characteristics, making feasible the long-term tracking of an individual's functional architecture by centralized systems. To index a patient's multi-echo fMRI report, capturing their functional state at a point in time for comparison cross-sectionally or longitudinally, an identifying mark such as a QR code or barcode is generated. This tag can be used by patients or providers to retrieve data related to the exam, including data processed and retained but not directly reflected in the report.

The eighth feature involves the inclusion of quality control metrics (232). A feature of an fMRI-based subject report is the quality of the data upon which aspects of the report are based. This sub-report may convey quantitative, qualitative, or pass/fail aspects of the underlying data. For example, average or regional temporal signal-to-noise ratio (tSNR, a measure of signal stability) can be reported, representing a high-level measure of fidelity of signals, and thereby of the derived results. tSNR values may also be reported more specifically to a network or region of interest. A quality measure such as tSNR alone or in combination with others can inform an analytic decision on the value of the data and the derivative results, such as pass or fail.

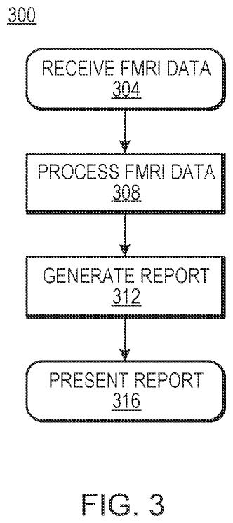

FIG. 3 is a flowchart of a method 300 for generating a multi-echo fMRI clinical report in accord with the present disclosure. For example, method 300 may be implemented by the apparatus 100 of FIG. 1. However, method 300 is not limited to that exemplary embodiment. Method 300 may include steps 304-312 as described below. Some of the steps may be optional to perform the disclosure provided herein.

In step 304 apparatus configured as described above may receive fMRI imagery associated with a patient, e.g., from imaging device 104 or storage 116.

In step 308 the apparatus processes the received fMRI imagery. The processing may include pre-processing the imaging data by, e.g., filtering it to reduce noise and artifacts and performing other operations designed to improve the usability of the images, such as resizing and contrast adjustment. The processing also includes the generation of data supporting one or more of the features described above and implemented in the report, i.e., representing the subject's functional network in R2*-based units (204^{N}), the generation of a distinct functional network map (208^{N}), the juxtaposition of increased activity and decreased activity states in the state map (212^{N}), the computation of bulk activation metrics (216^{N}), the computation of functional identity (220^{N}), the computation of patient-specific functional architecture (224), the computation of the report identifier (228), and the computation of quality control metrics (232).

In step 312 the report is generated using the data from Step 308. The report may be presented to an operator using, e.g., user interface 120. In some embodiments an operator may user the user interface 120 to select the features included in the report. The report may be generated and regenerated to include different combinations of features.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. The systems and methods involving hardware and software and/or functional parts therefore may be physically integrated into or housed inside or attached to another device, be it an imaging device, a stimulus or electrophysiological recording device, and patient audio device, etc. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for generating reports using multi-echo fMRI data, the method comprising:
receiving multi-echo fMRI data;
processing, using a processor, the multi-echo fMRI data to generate at least one feature, the at least one feature being a representation of the subject's functional network in R2*-based units, a functional network map, a bulk activation metric, a functional identity, a patient-specific functional architecture, a report identifier, or a quality control metric;
generating, using the processor, a report using the at least one generated feature; and
presenting the generated report.

2. The method of claim 1, wherein the multi-echo fMRI data is received from an imaging device.

3. The method of claim 1, wherein the multi-echo fMRI data is retrieved from a storage device.

4. The method of claim 1, further comprising preprocessing the multi-echo fMRI data to improve its usability.

5. The method of claim 1, wherein the functional network map is color coded to reflect specific brain states.

6. The method of claim 1, wherein the report identifier includes a bar code.

7. The method of claim 1, wherein the quality control metrics include at least one of a temporal signal-to-noise ratio or a contrast-to-noise ratio.

8. The method of claim 1, wherein the generated report is presented via a static report or user interface.

9. A system for generating reports using multi-echo fMRI data, the system comprising:
a communication interface for receiving multi-echo fMRI data;
a user interface; and
a processor configured to:
process the multi-echo fMRI data to generate at least one feature, the at least one feature being a representation of the subject's functional network in R2*-based units, a functional network map, a bulk activation metric, a functional identity, a patient-specific functional architecture, a report identifier, or a quality control metric;
generate a report using the at least one generated feature; and
present via the user interface the generated report.

10. The system of claim 9 further comprising an imaging device.

11. The system of claim 9 further comprising a storage device for storing multi-echo fMRI data.

12. The system of claim 9, wherein the processor is further configured to preprocess the multi-echo fMRI data to improve its usability.

13. The system of claim 9, wherein the generated functional network map is color coded to reflect specific brain states.

14. The system of claim 9, wherein the generated quality control metrics include a temporal signal-to-noise ratio.

15. A non-transitory computer readable medium storing instructions that are executable by a processing device, and upon such execution cause the processing device to perform operations comprising:
receiving multi-echo fMRI data;
processing, using a processor, the multi-echo fMRI data to generate at least one feature, the at least one feature being a representation of the subject's functional network in ΔR2* units, a functional network map, a bulk activation metric, a functional identity, a patient-specific functional architecture, a report identifier, or a quality control metric;
generating, using the processor, a report using the at least one generated feature; and
presenting, via a user interface, the generated report.

16. The non-transitory computer readable medium according to claim 15 wherein the operations further comprise receiving multi-echo fMRI data from an imaging device.

17. The non-transitory computer readable medium according to claim 15 wherein the operations further comprise retrieving multi-echo fMRI data from a storage device.

18. The non-transitory computer readable medium according to claim 15 wherein the operations further comprise preprocessing the multi-echo fMRI data to improve its usability.

19. The non-transitory computer readable medium according to claim 15 wherein the functional network map is color coded to reflect specific brain states.

20. The non-transitory computer readable medium according to claim 15 wherein the report identifier includes a bar code.
